(19) United States
(12) Patent Application Publication
(10) Pub. No.: US 2004/0258761 A1
(43) Pub. Date: Dec. 23, 2004

(54) Polymer-based microcapsules and nanocapsules for diagnostic imaging and drug delivery and methods for their production

(76) Inventors: Margaret A. Wheatley, Media, PA (US); Nikhil Dhoot, Cortland Manor, NY (US); Justin Lathia, Elizabethtown, PA (US); Dalia El-Sherif, King of Prussia, PA (US)

Correspondence Address: Licata & Tyrrell P.C., 66 East Main Street, Marlton, NJ, 08053, US

(21) Appl. No.: 10/805,704

(22) Filed: Mar. 22, 2004

Related U.S. Application Data
(60) Provisional application No. 60/456,666, filed on Mar. 20, 2003.

(51) Int. Cl. A61K 9/16; A61K 9/50

(52) U.S. Cl. 424/490; 264/4.1

(57) ABSTRACT

Methods for producing polymer-based microcapsules and nanocapsules for use in diagnostic imaging and delivery of bioactive compounds as well as targeted imaging and delivery to selected tissues and cells are provided. Compositions containing these microcapsules and nanocapsules for use in diagnostic imaging and delivery of bioactive agents are also provided. Methods for enhancing delivery of nanocapsules via ultrasound are also provided.

This patent application claims the benefit of priority from U.S. Provisional Application Ser. No. 60/456,666, filed Mar. 20, 2003, which is herein incorporated by reference in its entirety.

### INTRODUCTION

This invention was supported in part by funds from the U.S. government (NIH Grant Nos. HL052901 and CA52823). The U.S. government may therefore have certain rights in the invention.

### FIELD OF THE INVENTION

The present invention provides polymer-based microcapsules and/or nanocapsules for diagnostic imaging and drug delivery and methods for their production. The present invention also relates to methods for production of polymer-based ultrasound contrast agents which comprise a biocompatible, biodegradable polymer which can be loaded with a bioactive compound and/or a targeting moiety. In addition, the present invention provides methods for delivery of these nanocapsules alone or in combination with other agents including, but not limited to free drug, genetic material, non-echogenic capsules with or without drug payload, or combinations thereof. Methods are also provided for facilitating or enhancing delivery of nanocapsules to a selected tissue or tissues via vasculature and extravascular spaces too narrow for access with larger microcapsules, e.g. leaky tumor vasculature, using ultrasonic waves to force the nanocapsules through gaps in the vasculature and extravascular spaces by mechanisms including, but not limited to, cavitation and microstreaming.

### BACKGROUND OF THE INVENTION

Ultrasound contrast agents are used routinely in medical diagnostic, as well as industrial, ultrasound. For medical diagnostic purposes, contrast agents are usually gas bubbles, which derive their contrast properties from the large acoustic impedance mismatch between blood and the gas contained therein. Important parameters for the contrast agent include particle size, imaging frequency, density, compressibility, particle behavior (surface tension, internal pressure, bubble-like qualities), and biodistribution and tolerance.

Gas-filled particles are by far the best reflectors. Various bubble-based suspensions with diameters in the 1 to 15 micron range have been developed for use as ultrasound contrast agents. Bubbles of these dimensions have resonance frequencies in the diagnostic ultrasonic range, thus improving their backscatter enhancement capabilities. Sonication has been found to be a reliable and reproducible technique for preparing standardized echo contrast agent solutions containing uniformly small microbubbles. Bubbles generated with this technique typically range in size from 1 to 15 microns in diameter with a mean bubble diameter of 6 microns (Keller et al. 1986. J. Ultrasound Med. 5:493-498). However, the durability of these bubbles in the blood stream has been found to be limited and research continues into new methods for production of microbubbles.

Research has also focused on production of hollow microparticles for use as contrast agents wherein the microparticle can be filled with gas and used in ultrasound imaging. These hollow microparticles also have uses as drug delivery agents when associated with drug products. These hollow microparticles can also be associated with an agent which targets selected cells and/or tissues to produce targeted contrast agents and/or targeted drug delivery agents.

U.S. Pat. No. 5,637,289, U.S. Pat. No. 5,648,062, U.S. Pat. No. 5,827,502 and U.S. Pat. No. 5,614,169 disclose contrast agents comprising water-soluble, microbubble generating carbohydrate microparticles, admixed with at least 20% of a non-surface active, less water-soluble material, a surfactant or an amphiphillic organic acid. The agent is prepared by dry mixing, or by mixing solutions of components followed by evaporation and micronizing.

U.S. Pat. No. 5,648,095 discloses hollow microcapsules for use in imaging and drug delivery. The hollow microcapsules are made by combining a volatile oil with an aqueous phase including a water soluble material such as starch or a polyethylene glycol conjugate to form a primary emulsion. The primary emulsion then is combined with a second oil to form a secondary emulsion, which is hardened and allows for microcapsules to form around a liquid core of the volatile oil. The volatile oil is then removed by evaporation leaving a hollow microcapsule.

U.S. Pat. No. 5,955,143 discloses hollow polymer microcapsules that are produced by dissolving a film-forming polymer in a volatile non-aqueous solvent, dispersing into the polymer solution finely divided particles of a volatilizable solid core material, inducing formation of a solid polymer coating on the particulate solid core material to produce polymer microcapsules having an encapsulated solid core. This core is then removed to result in hollow microcapsules that can be then filled with gas for contrast imaging.

U.S. Pat. No. 6,521,211 describes ultrasound methods wherein the patient is administered a targeted vesicle composition and then scanned using ultrasound. The targeted vesicle composition comprises vesicles made up of a lipid, protein or polymer encapsulating a gas, in combination with a targeting ligand. Preferred vesicles are liposomes or micelles comprising a phospholipid such as dioleoylphosphatidylcholine, dimyristoylphosphatidyl-choline, dipalmitoylphosphatidylcholine, distearoyl-phosphatidylcholine, dipalmitoylphosphatidylethanolamine, dioleoylphosphatidylethanolamine, N-succinyldioleoyl-phosphatidylethanolamine, 1-hexadecyl-2-palmitoyl-glycerophosphoethanolamine, or a phosphatidic acid. Scanning is performed via dual frequency ultrasound insonation.

U.S. Pat. No. 6,416,740 discloses a method for the controlled delivery of a therapeutic compound to a region of a patient via administration of a targeted therapeutic delivery system comprising, in combination with a therapeutic compound, stabilized lipid microspheres encapsulating a gas or gaseous precursor and an oil. The therapeutic compound is encapsulated or embedded in the microspheres. Microspheres used in this method comprise at least one phosphatidylcholine, at least one phosphatidylethanolamine, and at least one phosphatidic acid. Examples of preferred phosphatidylcholines are dioleoylphosphatidylcholine dimyistoylphosphatidylcholine, dipalmitoylphosphatidylcholine, and distearoylphosphatidyl-choline. Examples of preferred phosphatidylethanolamines are dipalmitoylphosphatidylethanolamine, dipalmitoylphosphatidylethanolamine-PEG 5,000, dioleoyl-phosphatidylethanolamine, and N-succinyl-dioleoyl-phosphatidylethanolamine. A preferred phosphatidic acid is dipalmatoylphosphatidic acid. The presence of these microspheres in the region of the patient is monitored by diagnostic ultrasound. When present in the region, a therapeutic ultrasound is applied to the region to induce rupturing of the microspheres, thereby releasing the therapeutic compound in the region.

U.S. Pat. No. 6,478,765 describes an apparatus and methods for dissolving blood clots or other fistula obstructions using either a combination of ultrasonic energy and an echo contrast agent containing microbubbles or a selected dose of thrombolytic agent in combination with an echo contrast agent.

U.S. Pat. No. 6,139,819 discloses contrast agents for diagnostic and therapeutic uses comprising a lipid, a protein, polymer and/or surfactant, and a fluorinated gas, in combination with a targeting ligand. Such agents are particularly useful in imaging of an internal region of a patient suffering from an arrhythmic disorder.

Lanzi et al. in U.S. Pat. No. 5,690,907, U.S. Pat. No. 5,958,371, U.S. Pat. No. 6,548,046 and U.S. Pat. No. 6,676,963 disclose lipid encapsulated particles useful in imaging by x-ray, ultrasound, magnetic resonance, positron emission tomography or nuclear imaging which comprise a molecular epitope on the surface of the particle for conjugation of a ligand thereto.

U.S. Pat. No. 6,514,481 discloses nanosized particles referred to as “nanoclinics” for therapeutic and/or diagnostic use. These particles are made up of a core comprising a magnetic material such as ferrous oxide or ferric oxide, a silica shell surrounding the core with an outer diameter of less than 100 nm, and a targeting agent having specific affinity for a molecule on the surface of a target cell. The targeting agent is attached to the surface of the silica shell via a carbon spacer.

U.S. Pat. No. 6,485,705 discloses imaging contrast agents useful in ultrasonic echography comprising gas or air filled microbubble suspensions in aqueous phases containing laminarized surfactants and, optionally, hydrophilic stabilizers. The laminarized surfactants can be in the form of liposomes. The suspensions are obtained by exposing the laminarized surfactants to air or a gas before or after admixing with an aqueous phase.

U.S. Pat. No. 6,375,931 discloses gas-containing contrast agent preparations for use in ultrasonic visualization of a subject, particularly perfusion in the myocardium and other tissues, which promote controllable and temporary growth of the gas phase in vivo following administration. Therefore, these agents act as deposited perfusion tracers. The preparations include a coadministerable composition comprising a diffusible component capable of inward diffusion into the dispersed gas phase to promote temporary growth thereof. In cardiac perfusion imaging, the preparations may be coadministered with vasodilator drugs such as adenosine in order to enhance the differences in return signal intensity from normal and hypoperfused myocardial tissue, respectively.

U.S. Pat. No. 6,524,552 discloses compositions of matter useful in imaging cardiovascular diseases and disorders. The compositions have the formula V—L—R where V is an organic group having binding affinity for an angiotensin II receptor site, L is a linker moiety or a bond, and R is a moiety detectable in in vivo imaging of a human or animal body.

U.S. Pat. No. 6,315,981 discloses a contrast medium for magnetic resonance imaging comprising gas filled liposomes prepared by a method wherein an aqueous suspension of a biocompatible lipid is agitated in the presence of a gas at a temperature below the gel to liquid crystalline phase transition temperature of the biocompatible lipid until gas filled liposomes result. The gas used in this contrast medium is hyperpolarized rubidium enriched xenon.

U.S. Pat. No. 6,264,917 discloses targetable diagnostic and/or therapeutically active agents, e.g. ultrasound contrast agents, having reporters comprising gas-filled microbubbles stabilized by monolayers of film-forming surfactants, the reporter being coupled or linked to at least one vector.

However, there remains a need for microcapsules and nanocapsules and methods of production of microcapsules and nanocapsules used for contrast imaging and/or drug delivery.

### SUMMARY OF THE INVENTION

An object of the present invention is to provide a methods for producing polymer-based microcapsules and nanocapsules.

Another object of the present invention is to provide polymer-based microcapsules and nanocapsules produced in accordance with the methods of the present invention.

Another object of the present invention is to provide a contrast agent for diagnostic imaging in a subject which comprises polymer-based microcapsules and/or nanocapsules of the present invention that are filled with a gas. Such contrast agents may further comprise a targeting agent such as a peptide or antibody on the microcapsule and/or nanocapsule surface for targeting of the contrast agents to selected tissues or cells. Attachment of a targeting agent selective to a diseased tissue provides for a contrast agent which distinguishes between diseased and normal tissue. Use of contrast agents comprising the nanocapsules and/or microcapsules of the present invention permits imaging of tissues via access to locations of the vasculature too narrow for access via larger microcapsules, e.g. leaky tumor vasculature.

Another object of the present invention is to provide methods for imaging a tissue or tissues in a subject via administration of a contrast agent comprising polymer-based microcapsules and/or nanocapsules of the present invention that are filled with a gas. Contrast agents used in this method may further comprise a targeting agent such as a peptide or antibody on the microcapsule and/or nanocapsule surface for targeted delivery of the contrast agent to the selected tissue or tissues. Attachment of a targeting agent selective to a diseased tissue provides for a method of distinguishing via selective imaging diseased tissue from normal tissue. Similarly, attachment of a targeting agent selective to a malignant tissues provides for a method of distinguishing via selective imaging malignant tissue from benign tissue. Contrast agents of the present invention may be administered alone or in combination with additional agents including, but not limited to, free drug, genetic material, non-echogenic capsules with or without payload, or combinations thereof.

Another object of the present invention is to provide a composition for delivery of a bioactive agent which comprises a bioactive agent adsorbed to, attached to, and/or encapsulated in, or any combination thereof, polymer-based microcapsules and/or nanocapsules of the present invention. Such compositions may further comprise a targeting agent such as a peptide or antibody on the microcapsule and/or nanocapsule surface for targeting of the bioactive agent to selected tissues or cells. Attachment of a targeting agent selective to a diseased tissue provides for a delivery agent which delivers a bioactive agent selectively to diseased tissue. The bioactive agent can be released from the microcapsule and/or nanocapsule by exposure to ultrasound and/or upon degradation of the polymer-based capsule. Use of compositions comprising the nanocapsules and/or microcapsules of the present invention permits delivery of bioactive agents to locations of the vasculature too narrow for access via larger microcapsules, e.g. leaky tumor vasculature. Compositions of the present invention may be administered alone or in combination with additional agents including, but not limited to, free drug, genetic material, non-echogenic capsules with or without payload, or combinations thereof.

Another object of the present invention is to provide methods for delivery of bioactive agents to a subject via administration of a composition comprising a polymer-based microcapsule and/or nanocapsules of the present invention and a bioactive agent adsorbed to, attached to, and/or encapsulated in, or any combination thereof, the polymer-based microcapsule and/or nanocapsule of the present invention. Compositions used in this method may further comprise a targeting agent such as a peptide or antibody on the microcapsule and/or nanocapsule surface for targeting of the bioactive agent to selected tissues or cells in the subject. In this method, bioactive agent is released from the microcapsule and/or nanocapsule by exposure to ultrasound, degradation of the polymer-based capsule or a combination thereof. Compositions of the present invention may be administered alone or in combination with an additional agent such as, but not limited to, free drug, genetic material, non-echogenic capsules with or without drug payload, or combinations thereof.

Yet another object of the present invention is to provide methods for enhancing delivery of a bioactive agent to selected tissues via vasculature and extravascular spaces too narrow for access by larger microcapsules which comprises administering to a subject a composition comprising the bioactive agent adsorbed to, attached to, and/or encapsulated in, or any combination thereof, a nanocapsule, preferably a polymer-based nanocapsule of the present invention, and exposing the subject to ultrasonic waves which force the composition through small gaps of the vasculature and extravascular spaces too narrow for access via large microcapsules by mechanisms including, but not limited to, cavitation and microstreaming. Enhancing delivery to a targeted tissue by ultrasound is useful in drug delivery techniques involving the present invention as well as imaging techniques.

### DETAILED DESCRIPTION OF THE INVENTION

The present invention provides polymer-based microcapsules and/or nanocapsules and methods for producing such microcapsules and nanocapsules which are useful as imaging agents and in drug delivery. The microcapsules and nanocapsules of the present invention can be modified to be loaded with bioactive agents. Further, the microcapsules and nanocapsules of the present invention can be modified on their surface with a bioactive moiety that specifically targets the microcapsule and/or nanocapsule to selected tissue types. These microcapsules and nanocapsules of the present invention are capable of extravasation to specific tissues in areas such as a tumor and are capable of functioning as an ultrasound contrast agent. The nanocapsules and microcapsules of the present invention can also be used to carry and deliver a drug payload to a specific target in the body. Furthermore, these nanocapsules and microcapsules can be used to deliver the drug payload at a selected target through an ultrasound triggering mechanism and/or rate predetermined biodegradation.

Ultrasound can also be used to enhance delivery of nanocapsules such as those disclosed herein to selected tissues via holes in the vasculature and extravascular spaces too narrow for access by larger microcapsules, e.g. leaky tumor vasculature. In this method, a composition comprising the bioactive agent adsorbed to, attached to, and/or encapsulated in, or any combination thereof, a nanocapsule, preferably a polymer-based nanocapsule of the present invention is administered to the subject. The subject is then exposed to ultrasonic waves which force the composition through small gaps of the vasculature and extravascular space too narrow for access by large microcapsules via mechanisms including, but not limited to, cavitation and microstreaming. Enhancing delivery to a targeted tissue by ultrasound is useful in drug delivery techniques involving the present invention as well as imaging techniques.

The nanocapsules and microcapsules of the present invention comprise a biocompatible, biodegradable polymer. In a preferred embodiment, the polymer-based microcapsules or nanocapsules are loaded with a bioactive compound. Biodegradation of the polymer capsule proceeds at a rate predetermined by the choice of polymer and insonating frequency, providing a controlled release of the bioactive compound(s), and resulting in a controlled release of the compound over a predetermined time period.

The polymer-based nanocapsules or microcapsules of the present invention can be prepared in accordance with the following method. A biocompatible, biodegradable polymer is dissolved in a solution comprising an oil phase and a substance soluble in the oil phase and easy to sublime in the lyophilizer. If the oil phase is an organic solvent such as acetone, this sublimable substance may be camphor, ammonium carbamate, theobromide, camphene or napthalene. An emulsion of large beads or capsules of mixed polymer and a sublimable substance such as camphor is then formed in the solution by probe sonication. The resulting emulsion is poured into a surfactant solution, preferably a 1% solution of polyvinyl alcohol, and homogenized to remove the oil phase, for example acetone from the capsules, causing them to shrink in size. The addition of the surfactant allows the breakup of the polymer/sublimable substance beads or capsules into smaller ones, thus enhancing the size reduction of the capsules. The emulsion is then washed with deionized water to remove additional acetone and dry the capsules. The capsules are then collected by centrifugation, washed, and re-collected by centrifugation. The washed capsules are then frozen at −85° C. for approximately 30 minutes and dried, preferably by lyophilization to remove any additional sublimable substance.

Alternatively, microcapsules and/or nanocapsules of the present invention can be prepared by a double emulsion or w/o/w emulsion process. In the process, the sublimable substance such as camphor is dissolved with a biocompatible, biodegradable polymer such as PLA in an oil phase such as acetone. A first emulsion is then generated by addition of ammonium carbonate followed by sonication. This first emulsion (w/o) is then poured into a surfactant solution such as PVA and homogenized. The double emulsion (w/o)/w in then poured into water and stirred. Resulting capsules and collected via centrifugation, washed, and lyophilized variation in parameters such as sonication time, homogenization time and polymer blend as well as concentrations of ammonium carbonate alters the capsule size.

In one embodiment, a bioactive agent such as a drug is incorporated into the polymer-based nanocapsules or microcapsules of the present invention. Bioactive agents may be adsorbed to and/or attached to the surface of the nanocapsule and/or microcapsule. To adsorb a drug product to the nanocapsule or microcapsule surfaces, the drug is dissolved in distilled water or a buffer, and then the dried nanocapsules or microcapsules are suspended in distilled water with the drug. The suspension is stirred overnight and then centrifuged to collect capsules. The resulting nanocapsules or microcapsules are then washed, frozen and lyophilized. The lyophilized nanocapsules or microcapsules have the drug product to be delivered adsorbed to their surfaces. Bioactive agents can also be attached to the nanocapsules or microcapsules in accordance with well known methods for conjugation. For example, a conjugation method such as taught in Example 2 may be used substituting the bioactive agent for the peptide. Alternatively, or in addition, a bioactive agent can be encapsulated in the nanocapsules or microcapsules. Water soluble bioactive agents can be encapsulated in the nanocapsules or microcapsules by including water during emulsification and dissolving the bioactive agent in this water forming a w/o/w emulsion system. Further, a water soluble, lyophilizable agent such as ammonium carbonate or ammonium carbamate can be included in the water phase, to increase echogenicity of the agents. This is removed during freeze drying. Non-water soluble bioactive agents can be encapsulated in the nanocapsules by dissolving the bioactive compound in the non-polar organic solvent in the first step of preparation of these capsules. Examples of bioactive agents which can be adsorbed, attached and/or encapsulated in the microcapsules and/or nanocapsules of the present invention include, but are not limited to, antineoplastic and anticancer agents such as azacitidine, cytarabine, fluorouracil, mercaptopurine, methotrexate, thioguanine, bleomycin peptide antibiotics, podophyllin alkaloids such as etoposide, VP-16, teniposide, and VM-26, plant alkaloids such as vincristine, vinblastin and paclitaxel, alkylating agents such as busulfan, cyclophosphamide, mechlorethamine, melphanlan, and thiotepa, antibiotics such as dactinomycin, daunorubicin, plicamycin and mitomycin, cisplatin and nitrosoureases such as BCNU, CCNU and methyl-CCNU, anti-VEGF molecules, gene therapy vectors and other genetic materials and peptide inhibitors such as MMP-2 and MMP-9, which when localized to tumors prevent tumor growth.

The microcapsules and/or nanocapsules of the present invention may further comprise a targeting agent attached to the capsule surface, which upon systemic administration can target the contrast agent or the delivery agent to a selected tissue or tissues, or cell in the body. Targeting agents useful in the present invention may comprise peptides, antibodies, antibody fragments, or cell surface receptor-specific ligands that are selective for a tissue or cell. Examples include, but are in no way limited to, RGD which binds to αv integrin on tumor blood vessels, NGR motifs which bind to aminopeptidase N on tumor blood vessels and ScFvc which binds to the EBD domain of fibronectin. Accordingly, targeting agents can be routinely selected so that a contrast agent or delivery agent of the present invention, or a combination thereof, is directed to a desired location in the body such as selected tissue or tissues, cells or an organ, or so that the contrast agent or delivery agent of the present invention can distinguish between various tissues such as diseased tissue versus normal tissue or malignant tissue versus benign tissue. Targeted contrast and/or delivery agents can be administered alone or with populations of contrast agents and/or delivery agents of the present invention which do not further comprise a targeting agent.

Surface-modified, gas-filled, polymer-based nanocapsules and microcapsules that are made according to the above methods are useful in medical applications such as targeted imaging contrast agents for cancer or tissue perfusion because their size allows them to penetrate into most any tissue. Further, penetration of the nanocapsules can be enhanced by ultrasonic waves which force the nanocapsules through leaks of the vasculature and extravascular spaces and to their target tissue. For example, the ultrasonic waves can be tuned to interact with the contrast agent in such a way as to cause cavitation or microstreaming, both of which will aid in displacing the agent or contents thereof through gap junctions in the capillaries. The ultrasound beam is focused on an area of interest, for example a tumor. Nanocapsules can also be injected into the vascular system for parenteral administration, or directly into a tumor or organ for local delivery. The drug/bioactive payload can also be used to stimulate angiogenesis in situations where this is advantageous such as tissue engineering constructs and replacement implants in areas such as the hip, and damaged heart. Accordingly, these nanocapsules and microcapsules of the present invention are useful in targeted ultrasonic imaging, targeted ultrasonic drug delivery, cancer diagnosis, cancer detection, prostate evaluation, and evaluation promotion of angiogenesis for implants and other conditions.

The following nonlimiting examples are provided to further illustrate the present invention.

### EXAMPLES

### Example 1

Production of polymer-stabilized Nanocapsules

Camphor (0.002 grams) was dissolved in 5 ml of acetone. After the camphor was fully dissolved, 0.075 grams of polymer was added and the mixture was stirred until the polymer dissolved. The solution was probe sonicated the mixture for approximately 15 seconds to form an emulsion. This emulsion step resulted in the production of large beads of mixed polymer and camphor. The emulsification was poured into 100 ml of a 1% PVA (polyvinyl alcohol) solution and homogenize for 7 minutes on 12,000 RMP to remove the acetone from the capsules, causing them to shrink in size. The addition of PVA (a surfactant) allowed the breakup of the polymer/camphor beads into smaller ones, thus enhancing the size reduction of the capsules. The emulsion was then poured into 100 ml of deionized water and stirred for 12 hours to further remove any acetone and dry the capsules. The capsules were then collected by centrifugation, washed with deionized water to remove surface PVA and centrifuged again to collect capsules after washing. Following the centrifugation, the capsules were frozen at −85° C. for 30 minutes. Following freezing, the capsules were dried and any additional camphor was removed by lyophilization.

### Example 2

Conjugation of Peptide to Polymer-Stabilized Contrast Agent

1-Ethyl-3-13-dimethylamino-propyl carbomiidie (EDC; 0.005 grams) and 0.0027 grams of N-hydroxysuccinimide (NHS) were dissolved in 10 ml of 2-[N-morpholino]ethanesulfonic acid (MES) buffer (pH 6.5). Nanocapsules (1 gram) from Example 4 were suspended in the mixture and shaken on a shaker for 15 minutes to activate the surface of the polymer and prepare the polymer for peptide attachment. Peptide (150μg) was added and the mixture was shaken for an additional 3 hours. Following shaking, the mixture was centrifuged to collect the capsules. The capsules were then washed with deionized H₂O and centrifuged again to collect capsules after washing. Collected capsules were frozen at −85° C. for 30 minutes and then lyophilized to dry the capsules and to remove any additional reagents.

### Example 3

Preparation of Nanocapsules by a w/o/w Emulsion Method

Camphor (0.004 g) and PLA (0.075 g) were dissolved in 5 ml of acetone. To generate the first (W/O) emulsion, 1.0 ml of 4% ammonium carbonate solution was added to the polymer solution and probe sonicated at 115 Watts for 30 seconds. The (W/O) emulsion was then poured into a 1% PVA solution and homogenized for 5 minutes at 9,500 rmp. The double emulsion (W/O)/W was then poured into pure water and stirred for 1 hour with a magnetic stirrer on a magnetic stir plate at a speed fast enough to create a vortex that spanned the entire solution. The capsules were collected by centrifugation for 5 minutes at 40,000 times g force, washed three times with hexane, then once with deionized water and lyophilized, using a Virtis Benchtop freeze dryer, to remove the camphor and ammonium carbonate core. The following experimental parameters were individually varied and their effects on capsule size observed: sonication time (15 s, 30 s), homogenization time (5 min, 7 min, 10 min), and polymer blend ratio of lactic to glycolic acid (LA:GA) (100:0, 85:15, 75:25). The concentrations of the encapsulating agents, ammonium carbonate (0%, 0.04%, 0.4%) and camphor (0 mg, 2 mg, 4 mg, 8 mg, 16 mg), were also varied and assessed for any resulting consequences on capsule size. Size was determined by dynamic light scattering and acoustic enhancement was determined by in vitro dose response analysis.

What is claimed is:

1. A method for producing polymer-based microcapsules or nanocapsules comprising:
(a) dissolving a biocompatible, biodegradable polymer in a solution comprising a sublimable substance and an oil phase;
(b) forming an emulsion of large capsules of mixed polymer and sublimable substance in the solution;
(c) pouring the emulsification into a surfactant solution to break-up the polymer/sublimable substance capsules into smaller capsules;
(d) removing the oil phase from the capsules, causing the capsules to shrink further in size to microcapsules and nanocapsules; and
(e) washing and collecting the microcapsules and nanocapsules.

2. A method for producing polymer-based microcapsules or nanocapsules comprising:
(a) dissolving a biocompatible, biodegradable polymer in a solution comprising a sublimable substance and an oil phase;
(b) adding ammonium carbonate to the solution of step (a);
(c) sonicating the solution of step (b) to form a first emulsion;
(d) pouring the first emulsion of step (c) into a surfactant solution;
(e) homogenizing the solution of step (d) to form a second emulsion;
(f) pouring the second emulsion of step (e) into water and stirring to produce polymer-based microcapsules and nanocapsules; and
(g) collecting and washing the produced polymer-based microcapsules and nanocapsules of step (f).

3. A microcapsule or nanocapsule produced in accordance with the method of claim 1 or 2.

4. A contrast agent for diagnostic imaging in a patient comprising microcapsules or nanocapsules of claim 3 filled with a gas.

5. The contrast agent of claim 4 further comprising a targeting agent attached to an outer surface of the microcapsules or nanocapsules.

6. A method for imaging a tissue or tissues in a subject comprising administering to the subject the contrast agent of claim 4.

7. A method for selectively imaging a tissue or tissues in a subject comprising administering to the subject the contrast agent of claim 5.

8. The method of claim 7 wherein the contrast agent selectively targets diseased tissue and distinguishes the diseased tissue from normal tissue.

9. The method of claim 7 wherein the contrast agent selectively targets malignant tissue and distinguishes the malignant tissue from benign tissue.

10. A composition for delivery of a bioactive agent comprising a microcapsule or nanocapsule of claim 3 and a bioactive agent adsorbed to, attached to, or encapsulated in, or any combination thereof, the microcapsule or nanocapsule.

11. The composition of claim 10 further comprising a targeting agent attached to an outer surface of the microcapsule or nanocapsule.

12. A method for delivering a bioactive agent to a subject comprising administering to the subject the composition of claim 10 and triggering release of the bioactive agent in the subject by ultrasound.

13. A method for delivering a bioactive agent to a subject comprising administering to the subject the composition of claim 10 wherein bioactive agent is released by degradation of the polymer-based microcapsule or nanocapsule.

14. The method of claim 13 wherein degradation of the polymer-based microcapsule or nanocapsule and release of the bioactive agent is altered by ultrasound.

15. A method for targeting a bioactive agent to a selected tissue in a subject comprising administering to the subject the composition of claim 11.

16. The method of claim 15 wherein the composition is targeted to diseased tissue.

17. The method of claim 15 wherein the composition is targeted to malignant tissue.

18. A method for enhancing delivery of a nanocapsule to a selected tissue via holes in vasculature too narrow for access via larger microcapsules comprising administering the nanocapsule to a subject and exposing the subject to ultrasonic waves which force the nanocapsule through holes in the vasculature.

19. A method for enhancing delivery of a nanocapsule to a selected tissue via holes in vasculature too narrow for access via larger microcapsules comprising administering a nanocapsule of claim 3 to a subject and exposing the subject to ultrasonic waves which force the nanocapsule through the holes in the vasculature.
